SEQUENCE LISTING (29 sequences)

SEQ ID NO: 1 - protein, 25 aa, Arabidopsis thaliana
MARKSLIFPVILLAVLLFSPPIYSA

SEQ ID NO: 2 - protein, 25 aa, Arabidopsis thaliana
MAGKSFMTPAIMLAMLLLISPETYA

SEQ ID NO: 3 - protein, 22 aa, Arabidopsis thaliana
MAKLSFTFCFLLFLLLSSIAAG

SEQ ID NO: 4 - protein, 26 aa, Glycine max
MAPNALFLFSVLAIPLLLLPCPTAAG

SEQ ID NO: 5 - protein, 24 aa, Glycine max
MAPNALFLFSFLAMLLLLLRSTAA

SEQ ID NO: 6 - protein, 28 aa, Glycine max
MASKFIFTTVVLLMLLCLLLMREPSGCG

SEQ ID NO: 7 - protein, 23 aa, Glycine max
MAMLTRFLGCICLILILSAGSEA

SEQ ID NO: 8 - protein, 25 aa, Oryza sativa
MGRLFLCLVVAWCWVALLLVAPVHG

SEQ ID NO: 9 - protein, 30 aa, Sorghum bicolor
MPAAAQSVRAMAAAAAVVAMLLSTAAVAAA

SEQ ID NO: 10 - protein, 33 aa, Oryza sativa
MLAASLRVEAVAVVAAAVLVLLLSPAAVVVVAG

SEQ ID NO: 11 - protein, 21 aa, Oryza sativa
MAAAGGAVLLLVLATATSVTG

SEQ ID NO: 12 - protein, 30 aa, Oryza sativa
MARRASIIVAAVIAACVLLVCMTTSSVVDA

SEQ ID NO: 13 - protein, 29 aa, Oryza sativa
MARRVGVVVAVIAACVLLVVGMTAPPVDA

SEQ ID NO: 14 - protein, 27 aa, Oryza sativa
MAARTYLAVFAFCAILMLSAVARSASA

SEQ ID NO: 15 - protein, 24 aa, Brachypodium distachyon
MASAAKAMAVVAATVLLLSTAAMA

SEQ ID NO: 16 - protein, 34 aa, Brachypodium distachyon
MAHAAAARSRCVVVAALAIAVAVFFLACLPPAAS

SEQ ID NO: 17 - protein, 24 aa, Brachypodium distachyon
MGRKACVLAMCLVLLLVLSSSVAS

SEQ ID NO: 18 - protein, 22 aa, Brachypodium distachyon
MARTRLGLFVCLVLLLSVAVHS

SEQ ID NO: 19 - protein, 31 aa, Populus trichocarpa
MARKSLTTSLRNLRVTAVPFLVLLSLHAVIG

SEQ ID NO: 20 - protein, 31 aa, Populus trichocarpa
MARKPLITFLGNFRVTAFLLLLLLTSRQVIG

SEQ ID NO: 21 - protein, 26 aa, Populus trichocarpa
MACPSKFYSLMLVLVLFYMVMEESYG

SEQ ID NO: 22 - protein, 26 aa, Populus trichocarpa
MALSFKFYSLIFLLGLLFMVLEESSG

SEQ ID NO: 23 - protein, 20 aa, Populus trichocarpa
MACVRFYLCVMLILLSFAQS

SEQ ID NO: 24 - protein, 21 aa, Solanum lycopersicum
MAPKYTSIIFLFLLFNSFSCS

SEQ ID NO: 25 - protein, 16 aa, Solanum lycopersicum
MAMILCIFLFSMQAQS

SEQ ID NO: 26 - protein, 26 aa, Solanum lycopersicum
MSLINAKYFNLFVLLICFLVIQESHG

SEQ ID NO: 27 - protein, 35 aa, Petunia hybrida
MSSSRVVTVSQDGTGDYRTVQEAIDTVPLSNRCRT

SEQ ID NO: 28 - DNA, 31 bp, Artificial Sequence
  UTR-Fw
tacatcacaatcacacaaaactaacaaaaga

SEQ ID NO: 29 - DNA, 30 bp, Artificial Sequence
  SacI-eNL-Rv
catggagctcttacgccagaatgcgttcgc

(19) United States
(12) Patent Application Publication
(10) Pub. No.: US 2021/0371477 A1
(43) Pub. Date: Dec. 2, 2021

(54) Photoprotein, Substrate Thereof and Use of Same

(71) Applicant: Osaka University, Osaka (JP)

(72) Inventors: Takeharu Nagai, Osaka (JP); Megumi Iwano, Osaka (JP)

(73) Assignee: Osaka University, Osaka (JP)

(21) Appl. No.: 16/972,138

(22) PCT Filed: Jun. 4, 2019
(86) PCT No.: PCT/JP2019/022198

§ 371 Date: Dec. 4, 2020

(30) Foreign Application Priority Data
Jun. 4, 2018 (JP) 2018-106866

(51) Int. Cl. C07K 14/415

(52) U.S. Cl. CPC C07K 14/415

(57) ABSTRACT

Provided is a method for causing a subject including a luminescent protein to emit light. The method provided is a method for causing a subject to emit light, wherein the subject is a plant body that has a fusion luminescent protein in a cell wall thereof, or a processed product of the same; the method includes the step of bringing a substrate composition into contact with the fusion luminescent protein; the fusion luminescent protein is a protein that includes a chemiluminescent protein moiety, a fluorescent protein moiety, and a moiety that connects the chemiluminescent protein moiety and the fluorescent protein moiety so that resonance energy transfer can occur from the chemiluminescent protein moiety to the fluorescent protein moiety; and the substrate composition contains a substrate of the chemiluminescent protein.

### TECHNICAL FIELD

The present disclosure relates to a luminescent protein, a substrate of the same, and use of these.

### BACKGROUND ART

Imaging of a living cell by using a fluorescent protein is a principal means for bioimaging. Excitation light is necessary to cause a fluorescent protein to emit light. However, there are such problems that the excitation light often has phototoxicity, that it causes the perturbation of light-dependent biological phenomena, and that it results in autofluorescence of a specimen.

These problems can be avoided by using a luminescent protein such as luciferase in place of the fluorescent protein. The luminescent protein can generate a radiation signal by catalyzing a (substrate) reaction involving a light emitting compound such as luciferin, and is completely independent from an external light source. However, a photon output having a greater intensity is required of a conventional luminescent protein.

To solve the dimness of the luminescent protein, attempts were made to improve intrinsic properties of luciferase such as the yield of light emission quantum, catalyst turnover, and stability. In recent years, NanoLuc (Nluc), which is the brightest luciferase, was developed by genetic engineering using Oplophorus luciferase (Oluc) of deep-sea shrimp, and furimazine as the optimal substrate was developed as well.

Nluc is very useful as having a high light emission intensity, but has a problem of poor color variation.

This problem was overcome by utilizing Forster resonance energy transfer (FRET) to a fluorescent protein that emits light of a target color. Rigid link between luciferase and a fluorescent protein enables light emission from an acceptor fluorescent protein owing to efficient FRET.

One of the examples thereof is Nanolantern. Renilla luciferase and a yellow fluorescent protein, Venus, were fused, whereby yellow Nanolantern (YNL) was created. Further, Renilla luciferase and an orange fluorescent protein, KusabiraOrange2, were bonded, whereby Orange Nanolantern (ONL) was created.

Further, a series of enhanced Nanolantern (eNL) was developed. Nluc was bonded with a cyan fluorescent protein, mTurquoise2, a green fluorescent protein, mNeonGreen, a yellow fluorescent protein, Venus, an orange fluorescent protein, mKOK, and a red fluorescent protein tdTomato, whereby cyan eNL (CeNL), green eNL (GeNL), yellow eNL (YeNL), orange eNL (OeNL), and red eNL (ReNL) were created, respectively (Non-patent Document 1).

Regarding luminescent proteins, to introduce the same into ornamental plants is proposed, as a purpose other than bioimaging. Patent Document 1 discloses that a plant into which luciferase is introduced is caused to absorb luciferin as a substrate through roots or cut stems of the same so that the plant emits light.

Further, Patent Document 2 discloses articles (toys, clothes, bath additives, etc.) that include a bioluminescent generation system.

### PRIOR ART DOCUMENT

### Non-Patent Document

[Non-Patent Document 1] Suzuki, K et al. Nat Commun. 2016, DOI: 10.1038/ncomms13718

### Patent Document

[Patent Document 1] JP-A-2006-42768
[Patent Document 2] JP-T-hei-11(1999)-504822

### SUMMARY OF THE INVENTION

### Problem to be Solved by the Invention

In the plant body disclosed in Patent Document 1, a luminescent protein is expressed in the cytoplasm. A substrate such as luciferin generally cannot pass through a cell membrane. In the case of Patent Document 1, therefore, the plant body is caused to absorb the substrate through roots or cut stems thereof.

However, an easy method is desired in a case where a plant body having a luminescent protein is used to emit light for ornament or entertainment purpose.

In light of this, the present disclosure, in one aspect, provides an easy method for causing a plant body having a luminescent protein to emit light.

### Means to Solve the Problem

In one aspect, the present disclosure relates to a method for causing a subject to emit light,

wherein the subject is a plant body that has a fusion luminescent protein in a cell wall thereof, or a processed product of the same,

the method including the step of bringing a substrate composition into contact with the fusion luminescent protein,

wherein the fusion luminescent protein is a protein that includes a chemiluminescent protein moiety, a fluorescent protein moiety, and a moiety that connects the chemiluminescent protein moiety and the fluorescent protein moiety so that resonance energy transfer can occur from the chemiluminescent protein moiety to the fluorescent protein moiety, and

the substrate composition contains a substrate of the chemiluminescent protein.

The present disclosure, in one aspect, relates to a plant body that has a fusion luminescent protein existing in a cell wall thereof, or relates to a processed product of the same, wherein the fusion luminescent protein includes: a chemiluminescent protein moiety; a fluorescent protein moiety; and a moiety that connects the chemiluminescent protein moiety and the fluorescent protein moiety so that resonance energy transfer can occur from the chemiluminescent protein moiety to the fluorescent protein moiety.

The present disclosure, in one aspect, relates to a recombinant fusion luminescent protein that includes: a signal sequence for a cell wall; a chemiluminescent protein moiety; a fluorescent protein moiety; and a moiety that connects the chemiluminescent protein moiety and the fluorescent protein moiety so that resonance energy transfer can occur from the chemiluminescent protein moiety to the fluorescent protein moiety.

### Effect of the Invention

According to the present disclosure, in one aspect, a method or an article with which a plant body that has a fusion luminescent protein can be caused to emit light can be provided.

### BRIEF DESCRIPTION OF DRAWINGS

FIG. 1 is a table showing exemplary cell wall signal sequences together with their plant species, protein names, and gene names.

FIG. 2 shows schematic diagrams illustrating vectors used for introducing genes of the fusion luminescent protein according to the present disclosure into a plant. One example of the cytoplasmic expression type, and one example of the cell wall expression type are shown.

FIG. 3 shows gene introduction of the fusion luminescent protein according to the present disclosure, which is an exemplary result confirmed by RT-PCR.

FIG. 4 shows locations where a fusion luminescent protein of the cytoplasmic expression type and that of the cell wall expression type, whose genes were introduced, were expressed; the locations are an exemplary result confirmed with a confocal laser microscope.

FIG. 5 shows exemplary expression intensities of a cytoplasmic expression type plant and a cell wall expression type plant when a substrate aqueous solution was brought in contact from outside (via leaves, etc.), the expression intensities being different between these plants.

FIG. 6 shows exemplary bright field images and luminescence images of plants of Arabidopsis thaliana into which the fusion luminescent protein of the cytoplasmic expression type and that of the cell wall expression type were introduced.

FIG. 7 shows exemplary fluorescence images of plants of Arabidopsis thaliana into which a fusion luminescent protein of the cytoplasmic expression type and that of the cell wall expression type were introduced.

FIG. 8 shows an example of a bright field image, a fluorescence image, and a luminescence image of plants of Arabidopsis thaliana into which a fusion luminescent protein of the cell wall expression type was introduced so as to be expressed specifically in petals.

FIG. 9 shows an example of a bright field image and a luminescence image of a plant of petunia into which a fusion luminescent protein of the cytoplasmic expression type was introduced.

FIG. 10 shows an example of a bright field image, a luminescence image, and a fluorescence image of a plant of petunia into which a fusion luminescent protein of the cell wall expression type was introduced.

FIG. 11 shows an example of a fluorescence image and a luminescence image of a plant of onion into which a fusion luminescent protein of the cell wall expression type was introduced.

### MODE FOR CARRYING OUT THE INVENTION

### [Fusion Luminescent Protein]

The luminescent protein used in the present disclosure or referred to in the present disclosure is a fusion luminescent protein that includes a chemiluminescent protein moiety, a fluorescent protein moiety, and a moiety that connects the chemiluminescent protein moiety and the fluorescent protein moiety so that resonance energy transfer can occur from the chemiluminescent protein moiety to the fluorescent protein moiety. In the present disclosure, the resonance energy transfer in one aspect is Forster resonance energy transfer (FRET).

In the fusion luminescent protein according to the present disclosure, the chemiluminescent protein moiety can function as a donor in FRET, and the fluorescent protein moiety can function as an acceptor in FRET.

### [Chemiluminescent Protein]

The chemiluminescent protein in the fusion luminescent protein according to the present disclosure, in one or a plurality of embodiments, is, for example, a bioluminescent protein, or a protein that catalyzes a light-generating reaction of a specific chemical substrate. Regarding one or a plurality of embodiments of the chemiluminescent protein, examples of the same include luciferase, aequorin, obelin, and combinations of any of these.

The chemiluminescent protein may be obtained by isolating or cloning luciferase, aequorin, and obelin existing in nature, or may be a chemiluminescent protein modification obtained by modifying the same. A modified chemiluminescent protein (chemiluminescent protein modification) in one or a plurality of embodiments is a chemiluminescent protein that has a mutation (e.g., deletion and/or substitution) in a part where the mutation does not influence characteristics of the chemiluminescent function (e.g., light emission intensity, emitted light color), and that has substantially identical or similar characteristics of the chemiluminescent function as compared with a state without the mutation. Alternatively, a modified chemiluminescent protein in one or a plurality of embodiments is a chemiluminescent protein that has a mutation (e.g., deletion and/or substitution) in a part where the mutation influences characteristics of the chemiluminescent function, and that has substantially different characteristics of the chemiluminescent function as compared with a state without the mutation.

In one or a plurality of non-limiting embodiments, examples of luciferase include sea pansy luciferase (Renilla luciferase), deep-sea shrimp luciferase (Oplophorus luciferase), sea firefly luciferase (Vargula/Cypridina luciferase), copepod luciferase (Gaussia luciferase, Metridia luciferase), dinoflagellate luciferase, luciferase of bioluminescent mushrooms (Mycena chlorophos, Omphalotus japinicus), luciferase of Sergia lucens, firefly luciferase (Photinus luciferase), bacterial luciferase, Akaluc, and Turboluc.

In the present disclosure, the “chemiluminescent protein moiety” refers to a chemiluminescent protein incorporated (fused) in a fusion luminescent protein, or a part of the same. The “chemiluminescent protein moiety” may be a whole of the chemiluminescent protein, or a part of the same. A chemiluminescent protein moiety in one or a plurality of embodiments is a part of the chemiluminescent protein that has a part of the N-terminal and/or the C-terminal deleted, and that has substantially identical or similar characteristics of the chemiluminescent function (e.g., light emission intensity, emitted light color) as compared with a state without the deletion.

As a chemiluminescent protein in the present disclosure, a commercially available chemiluminescent protein can be used.

### [Substrate]

In the present disclosure, the “substrate” refers to a substrate of a chemiluminescent protein incorporated (fused) in a fusion luminescent protein, or a substrate of a part of the same. The substrate of luciferase is called luciferin.

Generally, a chemiluminescent protein and a substrate are specifically combined, and persons skilled in the art can understand what is a substrate corresponding to a chemiluminescent protein (e.g., luciferin corresponding to luciferase), or can understand what is a chemiluminescent protein corresponding to a substrate (e.g., luciferase corresponding to luciferin).

Examples of the substrate in one or a plurality of non-limiting embodiments include firefly luciferin, bacterial luciferin, dinoflagellate luciferin, Vargulin, Coelenterazine, AlaLumine-HCl, substrates obtained by modifying these (modifications thereof), and combinations of any of these.

Firefly luciferin, bacterial luciferin, dinoflagellate luciferin, and Vargulin can become luciferin (substrate) for firefly luciferase, bacterial luciferase, dinoflagellate luciferase, and sea firefly luciferase (Vargula/Cypridina luciferase), respectively.

Coelenterazine can be a substrate of sea pansy luciferase (Renilla luciferase), deep-sea shrimp luciferase (Oplophorus luciferase), copepod luciferase (Gaussia luciferase), aequorin, and obelin.

The substrate may be obtained by isolating or synthesizing a substrate existing in nature, or may be a substrate modification obtained by modifying the same.

In the present disclosure, the “substrate composition” refers to a composition containing a substrate. The form of the composition may be liquid, or solid like a powder.

Generally, a substrate cannot pass through a cell wall. To deliver a substrate to a luminescent protein existing in cytoplasm, the following methods are available: a method of causing a plant to absorb the substrate through its root, its stem having a cut-away portion, or its leaf having a cut-away portion; and a method in which a surfactant is mixed in a substrate composition. In the case of the former method, time is required for a substrate to move from an absorbing part to a part to be caused to emit light. Besides, a substrate is consumed or decomposed in some cases. In the case of the latter method, a substrate composition can be applied directly to a part to be caused to emit light, but a plant body might be damaged by a surfactant.

According to the method of the present disclosure, a fusion luminescent protein exists in a cell wall of a subject to be caused to emit light (glow). Therefore, in one or a plurality of embodiments, even if no surfactant is contained in a substrate composition, the substrate composition may be applied or so to be into contact with a part to be caused to emit light, whereby the part can be easily caused to emit light.

In one or a plurality of non-limiting embodiments, the content of the surfactant in the substrate composition is equal to or less than a concentration at which the substrate can be delivered to a cytoplasm; or the content of the same is, for example, 1.0 mM or less, 0.5 mM or less, 0.1 mM or less, 0.01 mM or less, or 0.001 mM or less. Further, in one or a plurality of embodiments, the substrate composition may contain substantially less surfactant, or may contain no surfactant.

### [Fluorescent Protein]

Examples of fluorescent protein in the fusion luminescent protein according to the present disclosure include those that can function as an acceptor of energy of the above-described chemiluminescent protein functioning as a donor, and can absorb the energy of the donor to fluoresce. The fluorescent protein, in one or a plurality of embodiments, is a fluorescent protein that can increase the intensity of emitted light of the chemiluminescent protein, or a fluorescent protein that can change the color of the emitted light.

The fluorescent protein may be a fluorescent protein obtained by isolating or cloning a fluorescent protein existing in nature, may be a commercially available fluorescent protein, or may be a fluorescent protein modification obtained by modifying any of these. Fluorescent protein of various colors has been developed, and use of these makes it possible to variously design the color of emitted light of the fusion luminescent protein according to the present disclosure.

A modified fluorescent protein (fluorescent protein modification) in one or a plurality of embodiments is a fluorescent protein that has a mutation (e.g., deletion and/or substitution) in a part where the mutation does not influence fluorescence characteristics (e.g., light emission intensity, emitted light color), and that has substantially identical or similar fluorescence characteristics as compared with a state without the mutation. A modified fluorescent protein in one or a plurality of embodiments is a fluorescent protein that has a mutation (e.g., deletion and/or substitution) in a part where the mutation influences fluorescence characteristics (e.g., light emission intensity, emitted light color), and that has substantially different fluorescence characteristics as compared with a state without the mutation.

Examples of the fluorescent protein in one or a plurality of non-limiting embodiments include green fluorescent proteins, blue fluorescent proteins, cyan fluorescent proteins, yellow-green fluorescent proteins, yellow fluorescent proteins, orange fluorescent proteins, and red fluorescent proteins; In one or a plurality of more specific non-limiting embodiments, examples of the same include Azurite, bsDronpa, Cerulean, Citrine, Clover, CyOFP1, Dendra2, Dreiklang, Dronpa2, Dronpa3, DsRed, EBFP, EBFP2, ECFP, EGFP, Emerald, eqFP650, eqFP670, EYFP, Fast-FT, FusionRed, Gamillus, hmKeima8.5, IFP1.4, IFP2.0, iRFP670, iRFP682, iRFP702, iRFP713, iRFP720, Kaeda, KikGR, Kohinoor, LSSmCherry, LSSmKate2, LSSmOrange, mAG1, mAmetrine, mAmetrine1.2, mApple, mBlueberry2, mCardinal, mCerulean3, mCherry, mCherry2, mClover3, mCyRFP1, Medium-FT, mEOS2, mEOS3.1, mEOS3.2, mGarnet2, mIFP2, miniSOG, miRFP670, miRFP720, mKalama1, mKate2, mK-GO, mKOkappa, mKO1, mKO2, miris, mMaroon1, mNeonGreen, mNeptune, mNeptune2, mNeptune2.5, mNeptune681, mNeptune684, mOrange, mOrange2, mPlum, mRFP1, mRuby2, mRuby3, mScarlet, mScarlet-H, mScarlet-I, mStable, mStrawberry, mTagBFP2, mTFP1, mTurquoise, mTurquoise2, Neptune, NowGFP, oxFP series, Padron, PA-GFP, PAmCherry1, PAmCherry2, PAmCherry3, Phanta, PS-CFP, PS-CFP2, PSLSSmKate, PSmOrange, RDSmCherry1, rsCherry, rsEGFP, rsEGFP2, rsTagRFP, SBFP2, shyRFP, Sirius, skylan NS, skylan S, Slow-FT, SPOON, TagBFP, TagCFP, TagFP635, TagGFP, TagGFP2, TagRFP, TagRFP657, TagRFP675, TagRFP-T, TagYFP, T-Sapphire, TurboFP602, TurboGFP, TurboRFP, TurboYFP, UnaG, and Venus. For details of these fluorescent proteins, see, for example, https://sites.google.com/site/ilovegfp/Home/fps. In one or a plurality of more specific non-limiting embodiments, examples of the fluorescent protein include: a cyan fluorescent protein, mTurquoise2; a green fluorescent protein, mNeonGreen; a yellow fluorescent protein, Venus; an orange fluorescent protein, mKOK; and a red fluorescent protein tdTomato.

In the present disclosure, the “fluorescent protein moiety” refers to a fluorescent protein incorporated (fused) in a fusion luminescent protein, or a part of the same. The “fluorescent protein moiety” may be a whole of the fluorescent protein, or a part of the same. A fluorescent protein moiety in one or a plurality of embodiments is a part of the fluorescent protein that has a part of the N-terminal and/or the C-terminal deleted, and that has substantially identical or similar fluorescence characteristics as compared with a state without the deletion.

As the fluorescent protein in the present disclosure, a commercially available fluorescent protein may be used.

### [Linker]

The “moiety that connects the chemiluminescent protein moiety and the fluorescent protein moiety so that resonance energy transfer can occur from the chemiluminescent protein moiety to the fluorescent protein moiety” in the fusion luminescent protein according to the present disclosure, in one or a plurality of embodiments, can refer to a linker formed with one or a plurality of amino acids in a case where the chemiluminescent protein and the fluorescent protein are linked via the linker; or alternatively, in a case where the chemiluminescent protein and the fluorescent protein are directly linked, the forgoing phrase can refer to a peptide bond at the connecting part.

The linker can be selected so as to increase the efficiency of resonance energy transfer from the chemiluminescent protein moiety as a donor to the fluorescent protein moiety as an acceptor. In one or a plurality of embodiments, the linker has a length of, for example, 1 to 10, 1 to 5, 2 to 4, or 2 to 3 amino acid residues.

The order of fusion of the chemiluminescent protein moiety and the fluorescent protein moiety in the fusion luminescent protein according to the present disclosure is not limited particularly; the chemiluminescent protein moiety may be on the N-terminal side, or the fluorescent protein moiety may be on the N-terminal side. The fusion luminescent protein according to the present disclosure, in one or a plurality of embodiments, may have a tag protein fused at the N-terminal or the C-terminal.

Regarding one or a plurality of non-limiting embodiments of the fusion luminescent protein according to the present disclosure, examples of the same include enhanced Nanolantern (eNL) series including cyan eNL (CeNL), green eNL (GeNL), yellow eNL (YeNL), orange-yellow (OeNL), and red eNL (ReNL) (Suzuki, K et al. Nat Commun. 2016, DOI: 10.1038/ncomms13718).

Other examples of the fusion luminescent protein according to the present disclosure include BAF-Y, BARAC, ffLuc-cp156, GpNluc, OgNluc, LSSmOg, Antares, iRFP670-2-Luc8, and iRFP792.

### [Fusion Luminescent Protein of Cell Wall Expression Type]

The fusion luminescent protein according to the present disclosure, in one aspect, is a fusion luminescent protein of the cell wall expression type which is expressed so as to exist locally in a cell wall. The fusion luminescent protein in the present aspect, when being expressed in a plant, exists locally in a cell wall of the plant.

Regarding one or a plurality of embodiments of the fusion luminescent protein of the cell wall expression type according to the present disclosure, examples of the same include fusion luminescent proteins that have a signal peptide for a cell wall when being expressed.

When the fusion luminescent protein of the cell wall expression type in the present aspect exists in a cell wall of a plant body, access of a substrate from outside the plant body to the fusion luminescent protein is significantly improved. For example, only spraying a substrate-containing liquid over flowers, leaves, and stems causes liquid-sprayed parts to emit light (glow).

The present disclosure, in one aspect, therefore relates to a recombinant fusion luminescent protein that includes: a signal sequence for a cell wall; a chemiluminescent protein moiety; a fluorescent protein moiety; and a moiety that connects the chemiluminescent protein moiety and the fluorescent protein moiety so that resonance energy transfer can occur from the chemiluminescent protein moiety to the fluorescent protein moiety. The present disclosure also relates to a DNA having a base sequence that encodes the recombinant fusion luminescent protein, and relates to a vector that allows for the expression of the recombinant fusion luminescent protein or has the DNA.

In the recombinant fusion luminescent protein in the present aspect in one or a plurality of embodiments, the signal sequence may remain in the fusion luminescent protein existing locally in a cell wall, or does not have to remain therein.

Coelenterazine or other luciferins cannot pass through a cell membrane of a plant cell. To cause a substrate unable to pass through a cell membrane to reach the fusion luminescent protein existing inside the cell membrane (cytoplasm), the following process is needed in a case of a plant body: causing the plant to absorb the substrate through its root; or making a cut-away portion in its stem or leaf and causing the plant to absorb the substrate through the cut-away portion. These processes have a problem that it takes much time for the substrate to reach a flower or a leaf, and the substrate is consumed during the time.

In the present disclosure, as the “signal sequence for a cell wall” in one or a plurality of embodiments, a sequence of a signal peptide included in the protein existing locally in a cell wall can be used, examples of which include a signal peptide for a cell wall of an enzyme that is involved in cell wall synthesis or swelling.

A non-limiting example of the cell wall signal sequence is MARKSLIFPVILLAVLLFSPPIYSA (SEQ ID NO: 1), which is an extracellular transport signal sequence of cellulase (Gene Name: AT1G70710) of Arabidopsis thaliana. In another plant body, a cell wall signal sequence of an ortholog of this gene can be used.

As extracellular transport signal sequences also exist in other enzymes functioning in a cell wall, it is also possible to use the cell wall signal sequences.

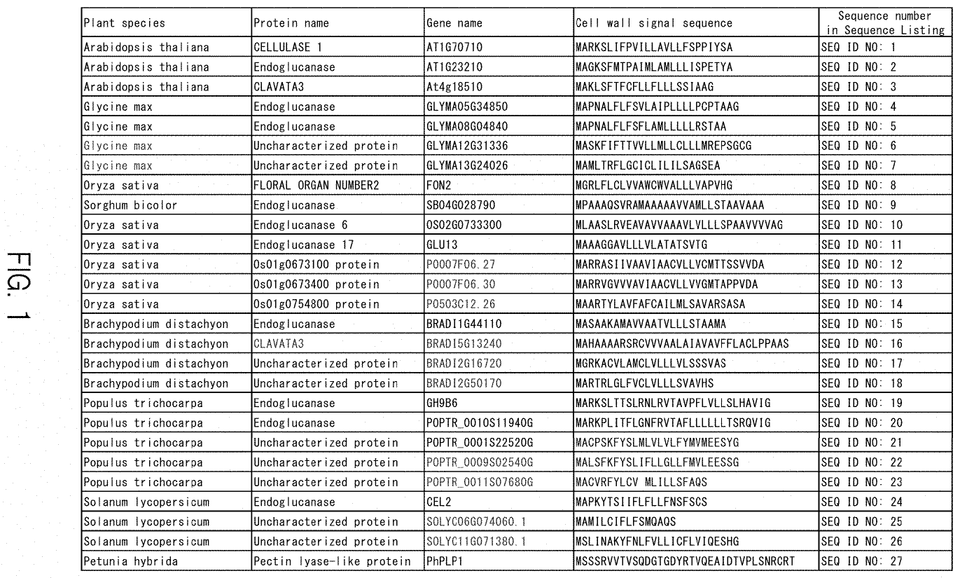

Non-limiting examples of species of plant bodies as well as proteins and genes having available signal sequences are shown in FIG. 1.

### [Subject]

A subject to be caused to emit light in the present disclosure is a plant body that has a fusion luminescent protein in a cell wall thereof, or a processed product of the same.

In the present disclosure, “has a fusion luminescent protein in a cell wall” in one or a plurality of embodiments can mean that a fusion luminescent protein exists at least in a cell wall; that a fusion luminescent protein unevenly exists more in a cell wall than in a cytoplasm; that a fusion luminescent protein exists specifically in a cell wall rather than a cytoplasm; or that a fusion luminescent protein exists locally in a cell wall.

In the present disclosure, “plant body” refers to a whole of a plant, or a part of the same, in one or a plurality of embodiments. “A part of a plant”, in one or a plurality of embodiments, refers to a flower, a petal, a corolla, a leaf, a stem, a root, and a combination of any of these.

“A processed product” of a plant in the present disclosure refers to an article including a plant body.

In one or a plurality of embodiments, a fusion luminescent protein may unevenly exist more in one or a plurality of parts of a plant body than in another part, may exist specifically in one or a plurality of parts of a plant body rather than in another part, or may exist locally in cell walls in one or a plurality of parts of a plant body. The part is, for example, a flower, a leaf, a stem, a root, and a combination of any of these, or a part of any of these. In one or a plurality of non-limiting embodiments, the plant body is a plant body in which a fusion luminescent protein exists locally in a cell wall of a corolla thereof, or a plant body in which a fusion luminescent protein exists locally in a cell wall of a leaf thereof.

In the present disclosure, examples of the plant include a plant for ornament, and a plant for materials, in one or a plurality of embodiments.

Examples of the plant for ornament include flowering plants and foliage plants. The plant for ornament is, for example, a plant cultured for the purpose of creating or maintaining beautiful appearance, or greening, and examples of the same include cut flowers (including bouquets) (rose, chrysanthemum, carnation, etc.); cut leaves (palm, etc.); cut branches (cherry, etc.); bulbs (tulip, lily, etc.); pot plants (cyclamen, orchid, foliage plants, dwarfed potted plants, etc.); nursery flowering plants (pansy, petunia, etc.); lawn; garden plants; and ground cover plants (bamboo grass, creeping plants, etc.).

Examples of the foliage plant include ivy (Araliaceae), maidenhair (Pteridaceae), Dypsis lutescens (Palmae), indian gum (Moraceae), Chlorophytum comosum (Liliaceae), sansevieria (Agavaceae), schefflera (Araliaceae), spathiphyllum (Araceae), dracaena (Agavaceae), neoregelia (Bromeliaceae), Pachira (Bombacaceae), Benjamin (Moraceae), pothos (Araceae), monstera (Araceae), and painted-leaf begonia (Begoniaceae).

Examples of the processed product of the plant for ornament may include cut flowers, cut leaves, cut branches, bulbs, pot plants, nursery flowering plants, lawn, garden plants made of plants for ornament. Examples of the potted foliage plant may include those of hydroculture, terrarium, and aqua terrarium.

Examples of the processed product of the plant for ornament may include “dead” plants for ornament such as herbaria (botanical specimens), preserved flowers, and dried flowers. Examples of the processed product of the plant for ornament may include accessories and interior objects made of plants for ornament.

Examples of the plant used as a material include trees. Examples of the processed product of the plant used as a material include woods and articles using the same.

In the present disclosure, examples of the plant include plant bodies of edible plants, in one or a plurality of embodiments.

### [Method for Causing Subject to Emit Light]

In one aspect, the present disclosure relates to a method for causing a plant body that has a fusion luminescent protein according to the present disclosure in a cell wall thereof, or a processed product of the same, to emit light, the method including the step of bringing a substrate composition into contact with the fusion luminescent protein.

In the method according to the present disclosure, the fusion luminescent protein, the plant body or the processed product of the same, and the substrate are as described above.

The contact of the substrate composition with the fusion luminescent protein can be achieved by, for example, spraying, atomizing, or applying a liquid substrate composition over a plant body that has a fusion luminescent protein according to the present disclosure in a cell wall thereof, or a processed product of the plant body. Alternatively, the plant body or the processed product of the same may be immersed in a liquid substrate composition.

As the fusion luminescent protein according to the present disclosure exists in a cell wall, the plant can be caused to emit light immediately after spraying or the like is performed.

The substrate in the liquid substrate composition used in the method according to the present disclosure can be used with the concentration thereof appropriately adjusted. The concentration of the substrate is set to or exceed, for example, a concentration that causes an enzyme reaction between the chemiluminescent protein (luciferase) moiety of the fusion luminescent protein and the substrate (luciferin) to have the maximum reaction rate.

In one or a plurality of embodiments, a plant body that has a fusion luminescent protein according to the present disclosure in a cell wall thereof can be produced by introducing genes of the fusion luminescent protein having a cell wall signal sequence into the plant body as a target, to obtain a transformant plant body.

As a method for obtaining a transformant plant body, a method that has been already reported and established can be used appropriately. The examples of the method include the agrobacterium method, the PEG-calcium phosphate method, the electroporation method, the liposome method, the particle gun method, and the microinjection method.

The agrobacterium method is performed with use of a protoplast, with use of a tissue slice, or with use of a plant body itself (the in-planta method). In the case where a protoplast is used, the agrobacterium method can be performed by co-culturing the protoplast with an agrobacterium having a Ti plasmid, by fusing the protoplast with a spheroplasted agrobacterium (the spheroplast method), or the like. In the case where a tissue slice is used, the agrobacterium method can be performed by infecting a sterile cultured leaflet (leaf disk) of a target plant with the protoplast, by infecting a callus with the protoplast, or the like. Alternatively, in the case where the in-planta method using a seed or a plant body is applied, i.e., in a system that does not involve culturing of a plant hormone-added tissue, the agrobacterium method can be performed by directly applying the agrobacterium to a water absorbing seed, an infant plant (seedling), a potted plant, etc. Therefore, the present disclosure in one aspect relates to a transformant plant body into which genes of a fusion luminescent protein of a cell wall expression type are introduced.

In the present aspect, the fusion luminescent protein of the cell wall expression type may exist locally in a cell wall of one or a plurality of parts of plant body. The part is, for example, a flower, a petal, a corolla, a leaf, a stem, a root, and a combination of any of these, or a part of any of these. In one or a plurality of embodiments, the fusion luminescent protein of the cell wall expression type can be caused to exist locally in a specific part by introducing genes of the fusion luminescent protein by utilizing an expression system or cassette of a site-specific expressed gene.

The fusion luminescent protein according to the present disclosure, in one or a plurality of embodiments, may be a recombinant protein produced by a gene recombination technique, or a chemically synthesized protein. The recombinant protein production by a gene recombination technique in one or a plurality of embodiments is achieved by a method in which a host transformed with an expression vector including a gene that encodes the fusion luminescent protein according to the present disclosure is used, or it is achieved with a non-cell system. The fusion luminescent protein according to the present disclosure may be formed with use of a tag protein or the like.

The present disclosure, in one aspect, relates to a plant body in which a fusion luminescent protein exists in a cell wall thereof, the fusion luminescent protein including: a chemiluminescent protein moiety; a fluorescent protein moiety; and a moiety that connects the chemiluminescent protein moiety and the fluorescent protein moiety so that resonance energy transfer can occur from the chemiluminescent protein moiety to the fluorescent protein moiety.

### [Nucleic Acid]

Therefore, the present disclosure, in one aspect, relates to a nucleic acid that encodes the fusion luminescent protein according to the present disclosure. In the present disclosure, exemplary nucleic acids include single-strand or double-strand DNAs selected from synthetic DNAs, cDNAs, genome DNAs, and plasmid DNAs, as well as transcription products of these DNAs.

### [Expression Cassette]

Therefore, the present disclosure, in one aspect, relates to an expression cassette that includes a nucleic acid encoding the fusion luminescent protein according to the present disclosure. In the expression cassette, an expression regulatory sequence corresponding to a host cell to be introduced is operatively connected to the nucleic acid. Exemplary expression regulatory sequences include a promoter, an enhancer, and a transcription terminator, as well as an initiation codon, a splicing signal of intron, and a stop codon.

### [Vector]

Therefore, the present disclosure, in one aspect, relates to a vector that allows for the expression of the fusion luminescent protein according to the present disclosure. In another aspect, the vector according to the present disclosure, in one or a plurality of embodiments, is an expression vector that includes a nucleic acid or an expression cassette according to the present disclosure. As the vector according to the present disclosure, an expression vector system corresponding to a cell in which the fusion luminescent protein is to be expressed (host) can be appropriately selected and used. Examples of the vector used as the vector according to the present disclosure, as one or a plurality of non-limiting embodiments, include plasmids, cosmids, YACS, virus (adenovirus, retrovirus, episome EBV, etc.) vectors and phage vectors, as well as binary vectors for the agrobacterium method.

### [Transformant]

The present disclosure, in one aspect, relates to a transformant that expresses the fusion luminescent protein according to the present disclosure. The disclosure, in one or a plurality of embodiments, relates to a transformant that includes a nucleic acid or a vector according to the present disclosure. The transformant of the present disclosure, in one or a plurality of embodiments, can be formed by introducing the nucleic acid, the expression cassette, or the vector according to the present disclosure into a host. Exemplary hosts include animal cells, plant cells, insect cells, and microorganisms.

The present disclosure also relates to one or a plurality of non-limiting embodiments described below:

[1] A method for causing a subject to glow,

wherein the subject is a plant body that has a fusion luminescent protein in a cell wall thereof, or a processed product of the same,

the method including the step of bringing a substrate composition into contact with the fusion luminescent protein,

wherein the fusion luminescent protein is a protein that includes a chemiluminescent protein moiety, a fluorescent protein moiety, and a moiety that connects the chemiluminescent protein moiety and the fluorescent protein moiety so that resonance energy transfer can occur from the chemiluminescent protein moiety to the fluorescent protein moiety, and

the substrate composition contains a substrate of the chemiluminescent protein.

[2] The method according to Item [1],

wherein the chemiluminescent protein is at least one luminescent protein selected from the group consisting of luciferase, aequorin, obelin, and combinations of any of these.

[3] The method according to Item [1] or [2],

wherein the substrate is at least one material selected from the group consisting of firefly luciferin, bacterial luciferin, dinoflagellate luciferin, Vargulin, Coelenterazine, and combinations of any of these.

[4] The method according to any one of Items [1] to [3],

wherein the fluorescent protein is at least one protein selected from the group consisting of green fluorescent proteins, blue fluorescent proteins, cyan fluorescent proteins, yellow-green fluorescent proteins, yellow fluorescent proteins, orange fluorescent proteins, and red fluorescent proteins.

[5] The method according to any one of Items [1] to [4],

wherein the bringing step is performed by spraying, atomizing, or applying the substrate composition over the subject.

[6] The method according to any one of Items [1] to [5],

wherein a plant of the plant body is a plant for ornament, a plant for materials, or an edible plant.

[7] The method according to any one of Items [1] to [6],

wherein the fusion luminescent protein exists locally in one or a plurality of parts of the plant body.

[8] A plant body in which a fusion luminescent protein exists in a cell wall thereof,

wherein the fusion luminescent protein includes:

a chemiluminescent protein moiety;

a fluorescent protein moiety; and

a moiety that connects the chemiluminescent protein moiety and the fluorescent protein moiety so that resonance energy transfer can occur from the chemiluminescent protein moiety to the fluorescent protein moiety.

[9] A recombinant fusion luminescent protein including:

a signal sequence for a cell wall;

a chemiluminescent protein moiety; a fluorescent protein moiety; and

a moiety that connects the chemiluminescent protein moiety and the fluorescent protein moiety so that resonance energy transfer can occur from the chemiluminescent protein moiety to the fluorescent protein moiety.

Hereinafter, although the following description describes the present disclosure in more detail by way of examples, these are illustrative, and the present disclosure is not limited to these examples.

### EXAMPLE

### Example 1

1. Construction of Vector for Introduction of Genes of Fusion Luminescent Protein of Cell Wall Expression Type into Plant Genome

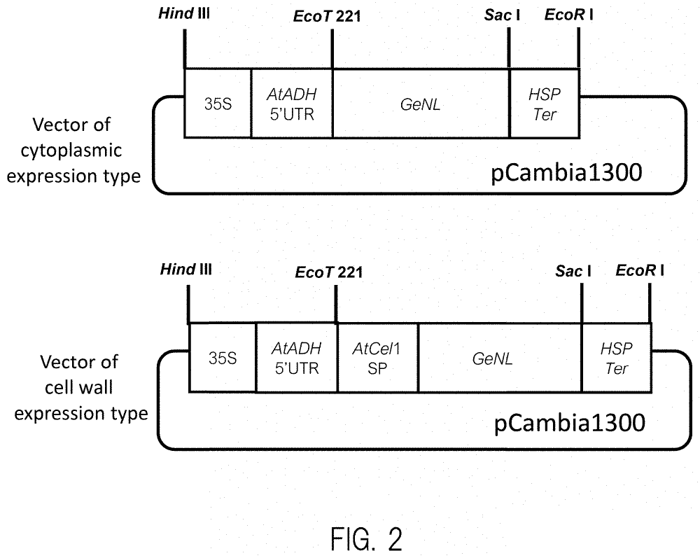

An expression cassette in which a protein of green enhanced Nanolantern (GeNL) fused with a cell wall signal peptide (AtCel1SP, SEQ ID NO: 1) of Arabidopsis thaliana was expressively connected with 35S promoter, Arabidopsis thaliana translation enhancer (AtADH5′-UTR), and HSP terminator was inserted into binary vector pCambia1300, whereby a vector for gene introduction that causes GeNL of the cell wall expression type to be expressed was constructed (FIG. 2). Similarly, a vector for gene introduction that causes cytoplasmic expression type GeNL having no cell wall signal peptide to be expressed was also produced (FIG. 2).

2. Introduction of Gene into Plant

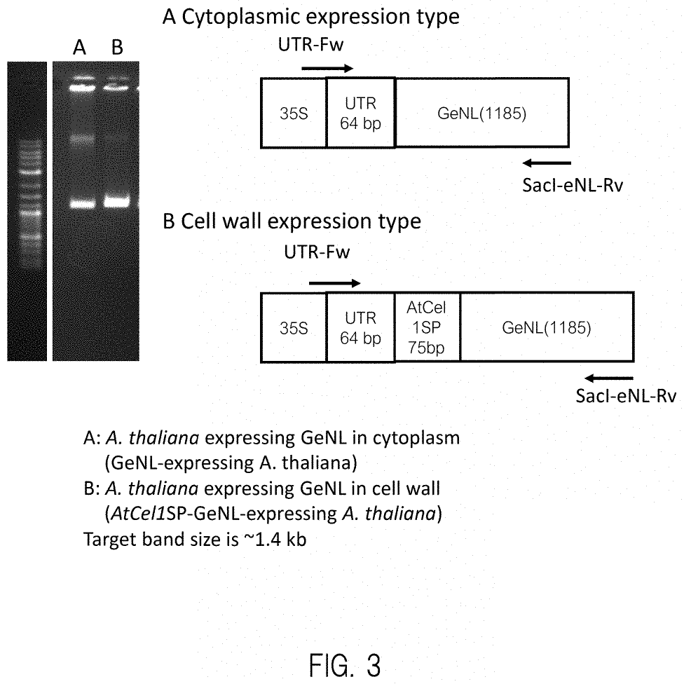

Vectors shown in FIG. 3 were introduced into Arabidopsis thaliana by the agrobacterium method. The expression of the fusion luminescent protein whose genes were introduced was confirmed by RT-PCR using the following primers and fluorescence emission (FIG. 3).

UTR-Fw:
(SEQ ID NO: 28)
TACATCACAATCACACAAAACTAACAAAAGA
SacI-eNL-Rv:
(SEQ ID NO: 29)
CATGGAGCTCTTACGCCAGAATGCGTTCGC

As shown in FIG. 3, the cell wall expression type GeNL was transcribed almost at the same level as that of the cytoplasmic expression type GeNL.

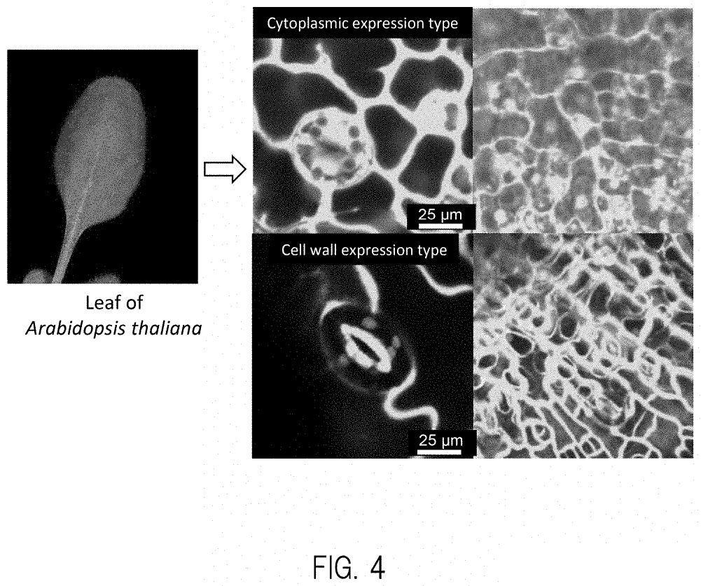

Next, expression sites of the fused luminescent protein in Arabidopsis thaliana, in which genes of the fused luminescent protein were introduced, were confirmed. Fluorescence of fluorescent protein moieties of the fusion luminescent protein was observed with a confocal laser microscope. The results are shown in FIG. 4.

As shown in FIG. 4, the expression of the cell wall expression type GeNL was recognized in cell walls. Further, under the same imaging conditions, fluorescence almost at the same level was observed regarding the cell wall expression type GeNL and the cytoplasmic expression type GeNL.

3. Differences in Light Emission when Substrate was Brought into Contact from Outside

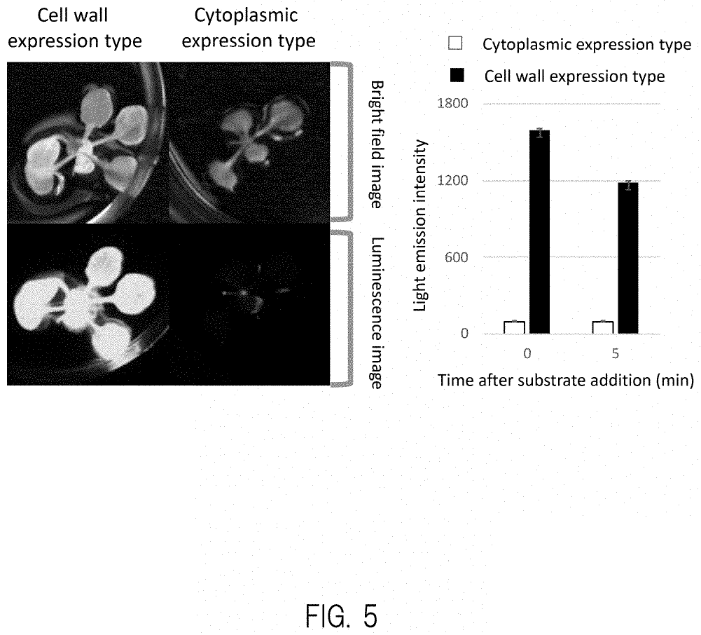

Plants of Arabidopsis thaliana into which the cell wall expression type GeNL or the cytoplasmic expression type GeNL was introduced were immersed in 50 μM of a substrate solution (Coelenterazine aqueous solution), and luminescence images were observed immediately and 5 minutes thereafter in a bright field and in a dark field. The results are shown in FIG. 5.

As shown in FIG. 5, in the case of the cell wall expression type, intense light emission was observed immediately after the contact with the substrate solution. On the other hand, in the case of the cytoplasmic expression type, intense light emission was not observed even at 5 minutes after the contact with the substrate solution.

It should be noted that more intense light emission was also observed from the plant of the cytoplasmic expression type having been brought into contact with a substrate solution to which a surface-active agent (1.5 mM, TritonX-100) was added, as compared with a case where it was brought into contact with a substrate solution containing no surface-active agent (results not shown).

### Example 2

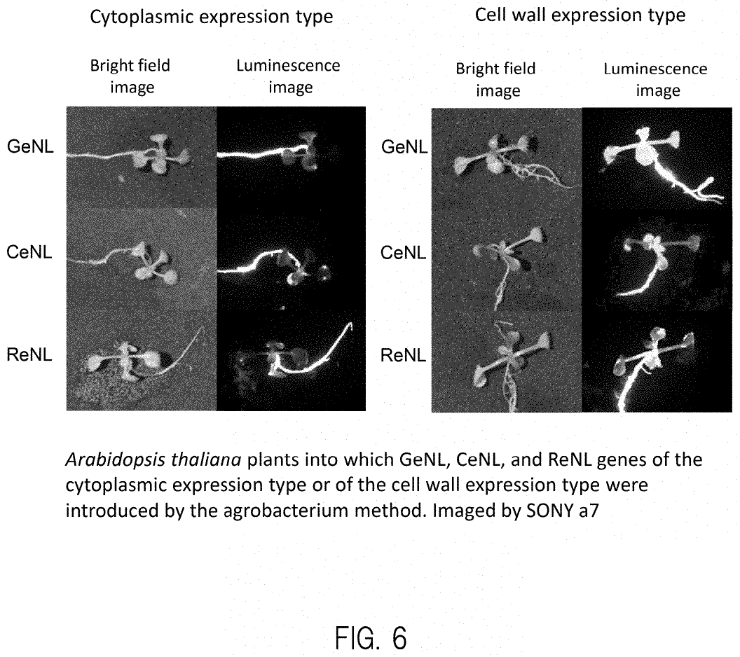

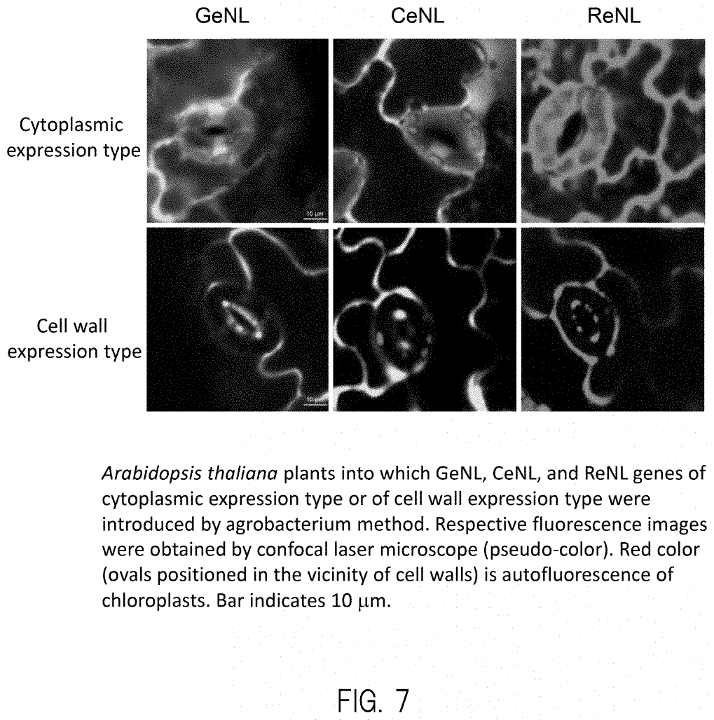

In addition to the transformants of GeNL, transformants were produced in the same manner as that in Example 1, by introducing constructs of CeNL or ReNL of the cytoplasmic expression type or of the cell wall expression type into plants of Arabidopsis thaliana. As a result of adding a substrate solution containing no surfactant, light emission was confirmed at root parts both in the case of the cytoplasmic expression type and in the case of the cell wall expression type. However, at leaves as parts on the ground, light emission was confirmed visually only in the case of the cell wall expression type (FIG. 6). In addition, it was confirmed that the fusion luminescent protein of the cell wall expression type thus introduced existed locally in cell walls and was expressed, by observation using a confocal laser fluorescence microscope (FIG. 7).

### Example 3

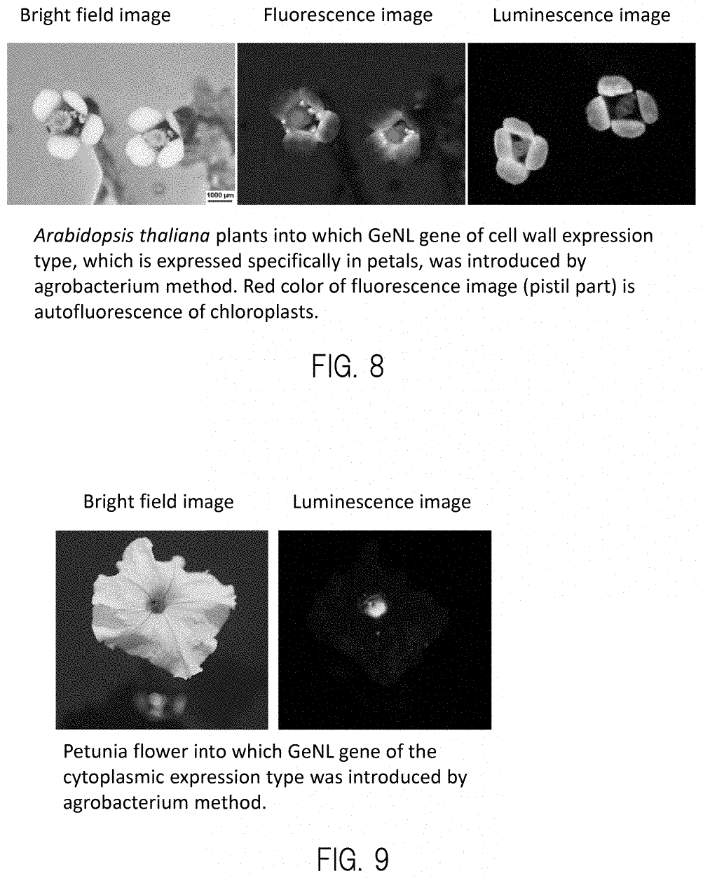

Gene Xygloglucan endotransglucosylase hydrolase 7 (XEH7), which is expressed specifically in petals of petunia, was identified, and the gene GeNL of the cell wall expression type was connected on the downstream side to the promoter, whereby a construct that causes GeNL to be expressed specifically in a cell wall of the petals was produced. This construct was introduced into Arabidopsis thaliana. As a result, with application of a substrate solution containing no surfactant onto flowers, light emission was confirmed visually (FIG. 8).

### Example 4

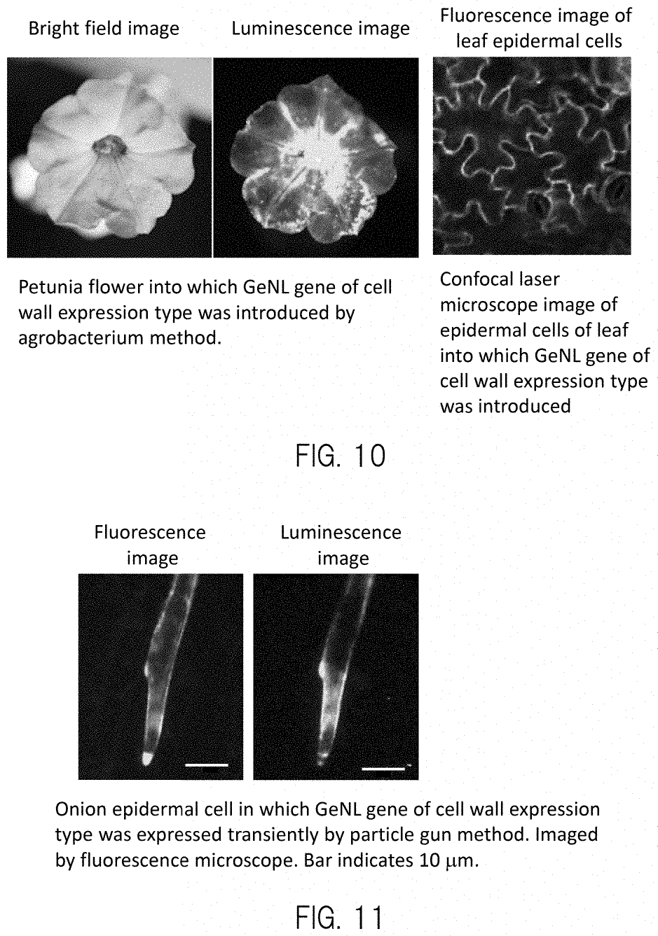

The GeNL constructs of the cytoplasmic expression type and the cell wall expression type were introduced in plants of petunia, and a substrate solution containing no surfactant was applied onto flowers; light emission was confirmed (FIGS. 9 and 10). As a result, the flowers (FIG. 10) of petunia into which the construct of the cell wall expression type was introduced exhibited more intense light emission as compared with those (FIG. 9) into which the construct of the cytoplasmic expression type was introduced.

### Example 5

A CeNL vector of the cell wall expression type was injected into plants of Nicotiana benthamiana by the agroinfiltration method (a method in which agrobacterium transformed with an expression vector is injected into leaves with a syringe, whereby a target gene is expressed transiently several days after). As a result, fluorescence of CeNL was confirmed, and with application of the substrate solution onto leaves, light emission was confirmed (data not shown).

### Example 6

A CeNL construct of the cell wall expression type was introduced transiently into plants of onion by the particle gun method (a method in which gold particles coated with an expression vector were inoculated into plant tissues with a particle gun, so that a target gene is expressed transiently). As a result, fluorescence of CeNL was confirmed in cell walls, and with application of the substrate solution, light emission was confirmed (FIG. 11).

### SEQUENCE LISTING

1. A method for causing a subject to emit light,
wherein the subject is a plant body that has a fusion luminescent protein in a cell wall thereof, or a processed product of the same,
the method comprising the step of bringing a substrate composition into contact with the fusion luminescent protein,
wherein the fusion luminescent protein is a protein that includes a chemiluminescent protein moiety, a fluorescent protein moiety, and a moiety that connects the chemiluminescent protein moiety and the fluorescent protein moiety so that resonance energy transfer can occur from the chemiluminescent protein moiety to the fluorescent protein moiety, and
the substrate composition contains a substrate of the chemiluminescent protein.

2. The method according to claim 1,
wherein the chemiluminescent protein is at least one luminescent protein selected from the group consisting of luciferase, aequorin, obelin, and combinations of any of these.

3. The method according to claim 1,
wherein the substrate is at least one material selected from the group consisting of firefly luciferin, bacterial luciferin, dinoflagellate luciferin, Vargulin, Coelenterazine, and combinations of any of these.

4. The method according to claim 1,
wherein the fluorescent protein is at least one protein selected from the group consisting of green fluorescent proteins, blue fluorescent proteins, cyan fluorescent proteins, yellow-green fluorescent proteins, yellow fluorescent proteins, orange fluorescent proteins, and red fluorescent proteins.

5. The method according to claim 1,
wherein the bringing step is performed by spraying, atomizing, or applying the substrate composition over the subject.

6. The method according to claim 1,
wherein a plant of the plant body is a plant for ornament, a plant for materials, or an edible plant.

7. The method according to claim 1,
wherein the fusion luminescent protein exists locally in one or a plurality of parts of the plant body.

8. A plant body in which a fusion luminescent protein exists in a cell wall thereof,
wherein the fusion luminescent protein includes:
a chemiluminescent protein moiety;
a fluorescent protein moiety; and
a moiety that connects the chemiluminescent protein moiety and the fluorescent protein moiety so that resonance energy transfer can occur from the chemiluminescent protein moiety to the fluorescent protein moiety.

9. A recombinant fusion luminescent protein comprising:
a signal sequence for a cell wall;
a chemiluminescent protein moiety;
a fluorescent protein moiety; and
a moiety that connects the chemiluminescent protein moiety and the fluorescent protein moiety so that resonance energy transfer can occur from the chemiluminescent protein moiety to the fluorescent protein moiety.
